(19) United States
(12) Patent Application Publication
(10) Pub. No.: US 2010/0139682 A1
(43) Pub. Date: Jun. 10, 2010

(54) Method and apparatus for precise deposition of hair care agents

(76) Inventors: Albert Durr Edgar, Austin, TX (US); David C. Iglehart, Austin, TX (US); Ralph Germer, Austin, TX (US); Douglas Yeager, Austin, TX (US); Rebecca Silvernail, Austin, TX (US); Rick B. Yeager, Austin, TX (US)

Correspondence Address: Galasso & Associates, LP, P.O. BOX 26503, Austin, TX, 78755-0503, US

(21) Appl. No.: 12/590,840

(22) Filed: Nov. 13, 2009

Related U.S. Application Data
(60) Provisional application No. 61/271,512, filed on Jul. 22, 2009.
(60) Provisional application No. 61/114,452, filed on Nov. 13, 2008.

(51) Int. Cl. A61Q 5/00; A61K 8/18; A45D 24/00

(52) U.S. Cl. 132/208; 132/221; 132/202; 132/163

(57) ABSTRACT

An apparatus and control method provides for automated, computer control to illuminate hair, sense aspects of that hair, calculate enhancements based on the sensed aspects of the hair, and precisely apply compounds on the hair in spatial conformance with the sensed aspects to create those enhancements. Examples of such compounds are hair coloring agents and hair care agents.

### RELATED APPLICATIONS

This U.S. patent application is related to the following patent applications, which are incorporated by reference into this application and hereby claimed priority to:
U.S. Provisional Patent Application No. 61,271,512 filed Jul. 22, 2009 by the inventor Albert D. Edgar, Douglas E. Yeager, David C. Iglehart, Rebecca Silvernail, Ralph Germer for “A DIGITAL BRUSH HAIR COLORING DEVICE”; and
U.S. Provisional Patent Application No. 61,114,452 Nov. 13, 2008 by the inventor Albert D. Edgar, Douglas Yeager, David C. Iglehart, Rebecca Silvernail, Ralph Germer, and Rick B. Yeager for “METHOD AND APPARATUS FOR PRECISE DEPOSITION OF HAIR CARE AGENTS”.

### BACKGROUND

1. Field of the Invention

The current invention relates to automated controlled methods that use sensors to selectively and precisely apply one or more hair care agent to living hair.

2. Background of the Invention and Prior Art

The current invention describes methods for the localized analysis and precise controlled deposition of agents, light energy, or heat energy on hair according to local characteristics of the hair. The methods and devices of the current invention facilitate both automation of existing hair treatment tasks such as hair coloring, and new approaches to hair treatment including more frequent application of milder hair color chemistries to maintain a desired look, enhancement of attractive natural variations in hair color and intensity, and the ability to monitor and extract detail at levels which are not detectable using conventional hair treatments. In addition to the precision delivery of hair coloring agents, the methods and devices of the current invention may be used to deliver other hair agents, such as conditioning and shine agents, and the precision delivery or monitoring of various wavelengths of light to support localized catalytic chemical reactions, localized hair straightening or curl, and fluorescent tagging as a secondary indicator of agent application for slow dyeing reactions.

### Comparison to Skin Beauty

Some of the methods and devices for hair analysis and the application of hair care agents correspond to previous work in skin beauty as described in copending U.S. patent application Ser. No. 11/503,806 filed Aug. 14, 2006 by applicants Albert D. Edgar, David C. Iglehart, and Rick B. Yeager for “SYSTEM AND METHOD FOR APPLYING A REFLECTANCE MODIFYING AGENT TO IMPROVE THE VISUAL ATTRACTIVENESS OF HUMAN SKIN” and U.S. patent application Ser. No. 12/129,624 filed May 29, 2008 by applicants Albert D. Edgar, David C. Iglehart, and Rick B. Yeager, for “APPARATUS AND METHOD FOR THE PRECISION APPLICATION OF COSMETICS” describe novel methods and devices for improving the visual attractiveness of human skin.

The '806 application describes Transparent Beauty™ which comprises a full skin area modeling and shaping with positional awareness and mapping. Image analysis is used in combination with an inkjet printer or other application device to selectively apply one or more transparent dyes in register in agreement or in register in opposition to local skin reflectance and surface characteristics.

The '624 application describes Eraser Brush™ which comprises a smoothing approach that does not require positional awareness. Image analysis is used in combination with a spray or other application device to selectively apply small amounts of pigment or other reflectance modifying agent when local skin properties exceed benchmark criteria.

### Theories of Beauty

Table 1 compares and contrasts embodiments of the current invention for hair care and development paths for skin beauty. Both the Transparent Beauty and the Eraser Brush were based on novel theories of skin beauty. Transparent Beauty was based on the observation that a more natural beauty can be obtained by selective deposition of surprisingly small amounts of transparent dyes. Eraser Brush was based on a similar observation that a more natural beauty can be obtained by sparse selective deposition of highly differentiated pigment. One aspect of these skin projects was that substantial improvements in appearance were possible by selectively filtering middle spatial frequency features.

One aspect of the current invention is that an appealing and natural look (“hairness”) can be obtained by selectively amplifying or moderating middle spatial frequency directional components.

Just as skin beauty is enhanced by naturally occurring high frequency features as opposed to a doll-like uniform surface, hair beauty may be enhanced through the deliberate manipulation of its spatial frequency directional components. For instance, the naturally occurring variations in brightness or color of strands of hair may be enhanced or muted independently along the length of the strands, across strands, and through the depth of the hair. Variations in brightness or color of strands of hair may be also artificially created in order to achieve different effects. Preferably, these variations are managed within middle spatial frequency ranges similar to those observed in skin.

It is of interest to note that there is a significant similarity between the process of evaluating and manipulating desired spatial frequency bands and the method used to process images in the human visual system. The visual system is highly sensitive to angle and spatial frequency variations because it, in effect, identifies components within the image by breaking it apart into different spatial frequency bands. Given this similarity to the method used to analyze both skin, for the case of Transparent Beauty, and now hair in the current, invention, it is logical that the visual attractiveness can be modified by selectively manipulating spatial frequencies.

### Photoshop™ Simulation

These theories of beauty and corresponding treatment protocols may be studied through powerful PhotoShop™ simulations. As summarized in Table 1, skin beauty theories were confirmed through robust PhotoShop simulation where the simulations were constructed in a manner that reflected physical constraints. For instance, the filtering parameters and deposition accuracies in the simulations were constrained to reflect hardware capabilities. Two types of results were obtained from these simulations. First, the resulting images from a wide range of skin type and skin tone provided a very effective demonstration of the theories—that the precise deposition of small amounts of various reflectance modifying agents did provide a previously unexpected, dramatic, and appealing natural beauty. Second, the simulations provided a powerful tool for evaluating control parameters and general control strategies. These parameters included reflectance modifying agent properties, deposition accuracy factors, number of passes, skin properties and other factors. For more advanced or dynamic simulations, PhotoShop simulations were combined with MatLab™ scripts.

As described in the embodiments below, simulation provides a similar powerful tool for both the visual confirmation of improved hair appearance and a tool for evaluating control parameters and general control strategies for hair beauty.

### Hardware Demonstrations

While the skin simulations were compelling, two there were two underlying concerns about the validity of simulation results. First, there was a general concern that skin was a complex three-dimensional surface that might not demonstrate the improved appearance of the simulated images. Second, there was a concern about whether the desired depositions were practical for available hardware components. For the skin projects, relatively simple hardware demonstrations were made before detailed prototype design. The Transparent Beauty approach was demonstrated with a commercial cake inkjet printer device, where a portion of a hand or forearm was imaged; manipulations of the captured image were performed by PhotoShop; and a desired corrective image was printed on the skin. For Eraser Brush, a commercial airbrush sprayer was adapted with a camera and control software to indicate whether a target region of skin was above or below a given threshold. Although these devices relaxed many eventual device constraints of size or speed, they were effective in validating the improved appearance of skin and in confirming the feasibility of developing commercial products based on these theories and deposition control strategies.

As described in the embodiments below, the commercial cake inkjet printer device, the adapted airbrush, other existing demonstration devices and new devices can be used to validate the theories of hair beauty and simulation results in order to demonstrate feasibility of developing commercial products based on these theories and deposition control strategies.

### Prototypes and Commercial Product Design

The hair projects are expected to follow the skin product development path of prototype devices and commercial product design.

The italicized portions of Table 1 indicate planned development steps.

TABLE 1
Comparison of Skin and Hair Beauty Development Paths

| | | | | Digital Brush |
|---|---|---|---|---|
| | | | | “full mapping” - |
| | | | Digital Brush | Second generation |
| | Transparent | | “combing” and | product-full hair |
| | Beauty- full skin | | “streaking” based | modeling, |
| | area modeling and | | on image analysis | coloring, and |
| | shaping with | | and threshold | shaping with |
| | positional | | control - | positional |
| | awareness and | Eraser Brush - | analogous to the | awareness and |
| | mapping | “smoothing” | Eraser Brush | mapping |
| Step 1 - Develop | A more natural | A more natural | An appealing and | An appealing look |
| novel theory of | beauty can be | beauty can be | natural look | can be obtained by |
| Beauty | obtained by | obtained by sparse | (“hairness”) can | a combination of |
| | selective | selective | be obtained by | selectively |
| | deposition of | deposition of | selectively | amplifying middle |
| | surprisingly small | highly | amplifying | spatial frequency |
| | amounts of | differentiated | various bands of | directional |
| | transparent dyes | pigment | middle spatial | components and |
| | | | frequency | positional shading |
| | | | directional | |
| | | | components | |

| Step 2 - Confirm | Extensive Photoshop simulation to | | Photoshop simulation to |
|---|---|---|---|
| theory through | (a) demonstrate results; and | | (c) demonstrate results; and |
| robust simulation | (b) evaluate parameters and control | | (d) evaluate parameters and |
| | strategies | | control strategies |
| Step 3 - Demonstrate | “cake printer” | EB Gen 1 - | Inkjet and spray deposition on hair and |
| preliminary | inkjet printing on | manual deposition | wig samples |
| hardware feasibility | small areas of skin | based on actual | Bench scale and simulation of key |
| and result | per external | camera images | hardware components |
| | Photoshop | | |
| | calculation | | |

### Overview of Conventional Approach to Selective Coloring of Hair

Traditional hair coloring treatments can be performed using several different methods, each of which involve trade-offs between time of application, degree of color variation within the hair, and cost of application. On one end of the spectrum, at-home hair coloring kits are used. These kits generally produce a color which has minimal variation within the hair, but offer the benefits of comparatively low cost and time of application. At the other end of the spectrum, hair treatments performed in salons can attempted to enhance the variation in color in the hair for either a more natural or more creative appearance, but the extent of variation is generally proportional to cost and time of application.

A second trade-off to that must be considered when coloring the hair is that between the permanence of the color deposited in hair and the amount of damage to which the hair is subjected. Permanent dyes use oxidative agents which break down components of the hair, including the cuticle and melanin molecules. However, there are semi-permanent and demi-permanent dyes available which are less permanent, but cause less damage. Customers also must consider the additional time and cost associated with using semi-permanent dyes which require more frequent reapplication. One aspect of the current invention is that its methods and devices facilitate the frequent use of these or specially-developed dyes.

A general application of hair colorant involves several steps. A first steps involves sectioning the hair as desired so that different colorants can be applied to produce variations in the hair. For example, at a salon, a weave is often created across hairs, creating streaks at a desired frequency. As an example, a ⅛^{th} inch weave is considered a high spatial frequency separation while a ¼″ inch weave or greater is considered a low spatial frequency separation. Once the hair is sectioned as desired, the colorant components, such as two components of an oxidative permanent hair dye system, are mixed and applied to the desired portions of hair. Following application of the wet chemistry, the customer must wait to allow the dye time to react as necessary and the color to form in the hair. This waiting period can range from 10 to 45 minutes. Finally, the excess material is rinsed from the hair and the sections are removed. Frequently, a conditioning agent is applied after rinsing the hair to help smooth the texture of the hair to disguise the damage that has occurred.

A detailed salon treatment may take an expert colorist applying 200 foils in 2 hours at a cost of $200 or more. This type of detailed process typically provides a resolution of about ⅛ inch “weave”. On the other hand, coarser treatments, such a ¼ inch weave provide a much less desirable appearance.

### Prior Art Devices for Hair Coloring

Over the years, numerous patent applications have been filed describing application devices which look like combs or brushes with hollow tines through which a colorant is expelled through holes at or near the tips. Other applications describe devices which look like a syringe with a single tip or a device with a soft tip like a marker pen or small brush. The described embodiments show the colorant expelled by pressure provided by hand or by motor. The amount of colorant expelled is controlled by the user. In an effort to not have “holidays”, areas which do not receive required colorant, the user typically applies too much colorant and applies it to most or all of the hair.

Although it may be possible for a subject to use the described devices on herself/himself to apply the colorant, as a practical and convenience matter, application by second person is more effective because it is very difficult for a user to precisely apply the colorant to the hair on the back of her head or other areas not readily seen. When too much colorant is applied, it is often very difficult for the user to pull the application comb through the hair.

Other applications or patents describe methods for salon or professional use to measure the color of hair before coloring. The user then decides what color results she wants, and the invention tells the user how to mix the colorant. Other publications describe methods for measuring the moisture content of hair. Others describe methods to measure hair characteristics using sophisticated spectroscopy technologies in the lab. While these methods may be useful to the professional beautician, they do not assist the subject to color her hair herself.

Conductive, inductive and pH sensors which can be used to measure moisture on the surface of a material are well known technologies.

Therefore, there is a need for the precise application of small amounts of hair coloring agents to color targeted, localized regions of hair through automated, computer control. Precise application means that the application is made to a specified location in the hair or to a specified a range of length in the hair. Moreover, it means that the amount of colorant applied is self limiting, because the applicator has a feedback loop in the device logic, so that when the system determines that the hair has received a sufficient quantity of the hair colorant, no more colorant is applied. The applicator is thus convenient and comfortable for the subject to use—especially when combing through the hair around the back of the head. In some embodiments, the device is configured that colorant is distributed in the hair by the combing or brushing movement of the device.

### The Aesthetic Importance of Digital Control

Since time immemorial, people have tried to improve their appearances by applying forms of hair colorants or other hair agents. Even in this age of computers, agents are still typically applied with brushes, sprays, and fingers, which are relatively blunt techniques that involve blanketing over relatively large areas of surfaces. This makes precise applications very difficult. For example, the special coloring of very small areas on and around hair roots, especially around the back of the head, is difficult to achieve manually, especially for the user on herself/himself.

The computerized, digital control of the application of hair colorants is particularly valuable for aesthetic reasons: small amounts of hair colorants may be applied exactly where they are needed, without blanketing an entire area. A small amount of hair color applied selectively to hair roots, at the pixel level, often looks better—more youthful, natural, and appealing—than a larger amount of hair colorants applied less effectively. As described below, computer control provides a capability to enhance or mimic desired natural variations in hair color and hair appearance.

The component technology for the digital control of the application of colorants currently exists, to be employed creatively through a digital brush. Components such as microchips, sensors, emitters, and fiber optics cables are available at low or moderate costs.

### BRIEF SUMMARY OF THE INVENTION

An aspect of the current invention is to demonstrate methods of improving hair beauty which can be explored and refined through simulation and delivered with a combination of image analysis and precise delivery components such as inkjet, precise spray devices, and specialized tine delivery systems.

An aspect of the current invention is to provide an apparatus and control method for the precise application of hair coloring agents to color hair roots through automated, computer controlled deposition of one or more hair coloring agent.

An aspect of the current invention is to provide an apparatus and control method for the precise application of hair coloring agents to color hair roots through automated, computer controlled deposition of one or more hair coloring agent.

An aspect of the current invention is the ability to precisely deposit one or more hair care agents. Another aspect of the current invention is the ability to precisely deposit one or more hair coloring agent in combination with one or more hair care agents.

An aspect of the current invention is the image analysis and control capability to precisely deposit one or more hair care agents with a digital comb or digital brush device as described in the related copending U.S. Provisional Patent Applications No. 61,012,473, No. 61,056,282, and No. 61,082,494.

An aspect of the current invention is the image analysis and control capability to use of one or more fluorescent dyes or encapsulated fluorescent dyes as an indicator of agent deposition in a multiple pass deposition device as described in copending U.S. Provisional Patent Application No. 61,082,494.

### BRIEF DESCRIPTION OF THE DRAWINGS

The following embodiment of the present invention is described by way of example only, with reference to the accompanying drawings, in which:

FIG. 1 shows the dimensions of low, middle, and high spatial frequency.

FIG. 2 is a representative diagram that illustrates an embodiment of a digital brush.

FIG. 3 is a representative diagram that illustrates an embodiment of a digital comb showing primary components such as a power supply, integrated circuit, and substrate reservoir.

FIG. 4 is a representative diagram of an embodiment design of a digital comb with separate emitting, sensing, and deposition tines.

FIG. 5, FIG. 6, FIG. 7, and FIG. 8 represent PhotoShop™ simulation results for different individuals.

FIG. 9 is a representative diagram that illustrates an embodiment of a digital brush with a camera and light sources mounted at the base of the brush and with tines to normalize the hair and deposit substrate.

FIG. 10 is a flow chart of one embodiment of simulation of the selective application of hair agents to smooth along hair strands and to amplify natural variations between hair strands.

### DESCRIPTION OF EMBODIMENT

### Digital Control Techniques

### Definitions

The term “strand” is defined as one or more hairs, and is typically that set of hairs guided between adjacent tines of a comb or brush.

The term “single step chemistry” is defined as any material which can produce its final effect in hair through a single chemical reaction with the hair.

The term “weave” is defined as the act of separating the hair, normal to the length of the hairs, to create strands of multiple hairs of a desired spatial frequency to which a hair treatment can be applied such that variations are created in the hair.

The term “holidays” is defined as a region of the hair that inadvertently does not receive adequate hair care agents due to inadequate detection.

The term “hair care agents” is defined as any compound, combination of compounds, or energy applied to the hair with the intent of altering its physical and/or optical properties.

The term “hair care compound” is defined as any substance, compound, combination of compounds, or energy applied to the hair with the intent of altering its physical and/or optical properties. For example, hair coloring agents and hair care agents.

The term “hair coloring agents” is defined as any substance that alters the optical properties of hair.

An “axis of interest” includes along a strand of hair, across one or more strands of hair, and through the depth of hair.

In the case of hair, the term “in agreement with” refers to the deliberate amplification of a measured attribute or attribute differential; and the term “in opposition to” refers to the deliberate suppression of a measured attribute or attribute differential.

In this specification the phrases “digital brush” and “digital comb” are used interchangeably.

### Spatial Frequencies

In the current invention, the term “spatial frequency” is defined as attributes measured over distances along or across strands of hair. Typical attributes include various components of perceived color, shine and lightness. The degree of variation within a region of any of these components will be referred to as the “energy” of that component over the region of hair.

In the current invention, the term “middle spatial frequency” is defined as attributes measured over distances along or across strands of hair in the range of approximately 0.04″ to 0.40″. This range is somewhat arbitrary and is selected based on its proven results in both skin and hair simulations. The range may be moved larger or smaller and narrower or broader without departing from the scope of the current invention.

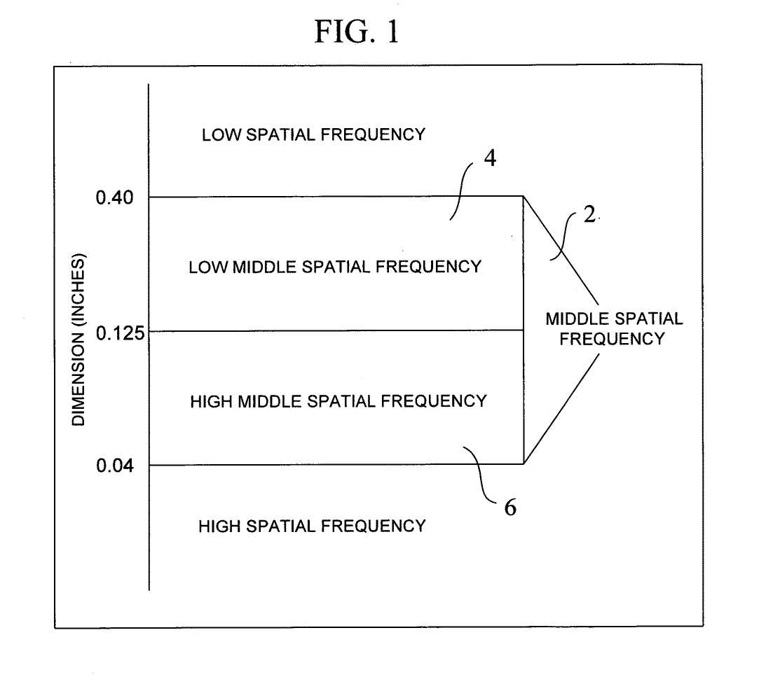

As illustrated by FIG. 1, it is useful in hair analysis to consider various bands of the middle spatial frequency 2. For instance, the term “low middle spatial frequency” 4 is defined as attributes measured over distances along or across strands of hair in the range of approximately 0.125″ to 0.40″; and the term “high middle spatial frequency” 6 is defined as attributes measured over distances along or across strands of hair in the range of approximately 0.04″ to 0.125″. In some cases it may be desirable to consider 3 or more distinct bands within the middle spatial frequency.

In the current invention, the term “low spatial frequency” is defined as attributes measured over distances greater than 0.40″.

### Methods for Enhancing Hair Appearance

### Overview

In one embodiment of the current invention, at least one optical sensor is used to obtain information regarding variations in hair and a fluorescent material is added to the hair colorant. The Digital Brush™ contains a means of illuminating the hair and sensing the amount of fluorescent material locally present near a sensor, as well as sensing the localized color properties of the hair. The measurements of hair variation are used to determine where to locally deposit a hair treatment product. Such an embodiment comprises at least one of each of the following: power source, reservoir for hair treatment compound(s), processor, light emitter, sensor, and deposition pathway for locally delivering hair treatment product colorant. Additionally, the embodiment may also comprise: a handle for ergonomic comfort, one or more detachable and replaceable components, a port or plug for recharging the power supply, one or more optical lenses to focus the emitter as desired, an on/off button for user control, and an electro-mechanical valve or pressurized gas cylinder to provide force for the deposition of colorant.

### Fluorescent Material

In one embodiment, at least one fluorescent tracer material is suspended within a hair treatment product such that the emission wavelengths of the material are detected by the sensor(s) and/or camera(s) as a means of determining whether or not a desirable amount of material has already been deposited in a localized region of the hair.

The fluorescent tracer can be excited by any wavelength of light. Due to health and cost considerations, however, it is most desirable to have an excitation peak which falls within the long wave ultraviolet or blue wavelengths, roughly 300-500 nm. It is desirable for a fluorescent tracer to have a distinct separation between excitation curve(s) and emission curve(s) so that an optical sensor can accurately detect only the photons emitted from the fluorescent tracer and not those reflected or coming directly from the excitation light source. Given the ideal excitation wavelengths listed above, it is desirable for the emission peak to fall within the green, red, or near-infrared wavelengths, most favorably red. An additional benefit to using a fluorescent tracer with an emission maximum within the red or infrared light ranges is that the hair is more transparent at these longer wavelengths than at shorter ones.

The fluorescent indicator should also have a short enough relaxation time to allow the emitter to be rapidly pulsed so that the emitter and fluorescent material remain undetected by the naked eye without impacting the ability of the material to undergo excitation and relaxation with each pulse. Rapidly pulsing an excitation source and tuning the sensor to the pulse frequency allows the capability to effectively eliminate ambient light at all other frequencies during the sensing, thereby reducing the amount of noise in the measurements.

Because the primary function of the fluorescent tracer is to provide an indication of the presence and or quantity of a hair treatment chemistry within the hair while using an optical system on an applicator device, it is not necessary for the tracer to remain in the hair after the application has been completed, and it is essential that the material not react with other components of the hair dye chemistry. This means that the fluorescent material does not need to bind to the surface of hair or penetrate into the cortex. It is generally accepted that pigments over 10 A are incapable of penetrating the cuticle of a hair. To ensure that the fluorescent material does not bind to the hair, it may be desirable to encapsulate the fluorescent in a shell that is optically clear and not reactive with the surface of hair.

In an embodiment that uses two optical sensors, it is possible to normalize for the distance of material detected and background brightness. For example, if the fluorescent material has an emission in the red wavelength range, and the sensors independently measure both the amount of red fluorescent light and the amount green light directly reflected from an emitter at a given point in time during an application process, the green measured green light can be used as a means of normalizing. Dividing the detected red by the detected green provides data for a more accurate measurement about how much fluorescent is present in the hair because the division helps to account for the distance that the material is away from the sensor and the background brightness of the system.

### Appearance

### Disadvantages of Conventional Hair Treatments

Conventional hair coloring processes are faced with several challenges which remain inadequately addressed, both through the use of at-home hair coloring kits and salon treatments. A dominant challenge with hair coloring is that the result appears unnatural as a result of the inability of a treatment to produce adequate variation in the hair. Rather, the dye tends to form a more monochromatic look, particularly if a single dye is used, as is generally the case with home use kits.

Hair coloring treatments applied at a salon generally result in a trade-off between a more natural looking-color variation and the time, expense, and amount of damage caused by an application session. Due to a lack of precision sensing in conventional hair coloring applications, there is little ability to enhance existing variations in the hair. Instead, a stylist can only mimic or attempt to recreate natural-looking variations by adding streaks and/or highlights of a desired size and color.

With most salon treatments, efforts to establish variation in hair generally results in an emphasis on the creation of variations across hairs, but there is generally little variation intentionally created along the length of hair. However, upon close inspection of untreated hair, it is clear that there is natural variation along both of these axes and that some of this variation is a result of sun exposure and other damage.

### Ability of the Current Platform to Address the Limitations of Conventional Hair Treatments

The current invention provides a means of detecting and modifying preexisting patterns and variations in the hair as desired. Whereas conventional permanent hair coloring application processes involve a gross application of oxidizing agents which break down the color-producing melanin molecules within the cortex, and semi-permanent hair coloring procedures bind to the surface of hair to mask the preexisting color, the current invention eliminates the need for a blanketing approach to coloring hair. With each of these conventional coloring techniques, there is a step which essentially masks the original color of the hairs, eliminating or greatly reducing variation. The current invention, however, allows a home user, or stylist, to selectively apply materials such that they enhance or attenuate existing color variations. Rather than first creating a blank canvas on which variations must be created from scratch, the natural variations can be selectively altered.

The ability to modify existing patterns in hair rather than first eliminating them and then attempting to recreate modified versions is analogous to the functionality of Transparent Beauty concepts for applying reflectance modifying agents to skin. In the case of Transparent Beauty, the selective detection and modification of specific spatial frequencies, for example the lightening of dark middle spatial frequencies, replaced the conventional approach of applying a foundation to eliminate variation at all spatial frequencies followed by the use of such agents as blush to reproduce lower frequencies. Thus the current invention parallels in hair the capability of the technology associated with Transparent Beauty to identify and selectively apply a substrate to a local region of skin.

Different embodiments of the current invention can be established to measure the optical properties of hair in one, two or three dimensions, as described above. Based on the physical characteristics of the brush applicator, it is possible to determine ranges of color intensity and spatial frequency to enhance and/or suppress independently along each of the measured axes. For example, it may be favorable to suppress middle frequency variation along the length of the hair but to attempt to manage the middle spatial frequencies measured across hairs by seeking to create approximately equal magnitudes of high-middle spatial frequency and low-middle spatial frequency such as discussed in the simulations below.

The ability to independently measure variations along the different measured axes and deposit hair treatment substrates based on evaluation of optical properties measured along multiple different axes can be used to provide new opportunity for selectively altering and/or creating the appearance of hair. As previously noted, the primary focus of stylists when treating hair is variation across hairs. However, by using a device that can measure natural variations on multiple axes, there is an enhanced ability to modify the optical effects of hair through a controlled deposition device.

### Signal-to-Noise Issues

One perspective of the current invention is that it provides a means to increase the signal-to-noise ratio of naturally occurring patterns. In the case of skin, high and low pass filtering techniques provide the ability to remove undesirable middle spatial frequency features—thereby removing “noise”. This approach increases the signal-to-noise ratio which the human eye perceives by reducing the noise on the middle frequency so that desirable natural high frequency variations are more noticeable.

In the case of hair, one approach to the treatment of signal-to-noise ratio is to analyze the hair dimensions separately. For instance, a filter may be performed on hair along the length of a strand in order to remove the “noise” along the length of the strand. Then, differences between adjacent strands can be analyzed and attenuated, such as by a factor of 2×-3× in order to amplify the “signal” of desirable high frequency variations. Thus the signal-to-noise ratio is increased by both a reduction in noise and an increase in signal.

Logically, we understand that it is important to have variations in hair color as opposed to a completely uniform appearance. It follows that the most desirable variations either (a) are completely random, or (b) follow at least some pattern for a particular individual. The digital analysis and controlled agent deposition of the current invention provide an ability to enhance the hair appearance in either case by invoking and/or enhancing random variations; or by using image analysis techniques to detect and enhance natural patterns of variation.

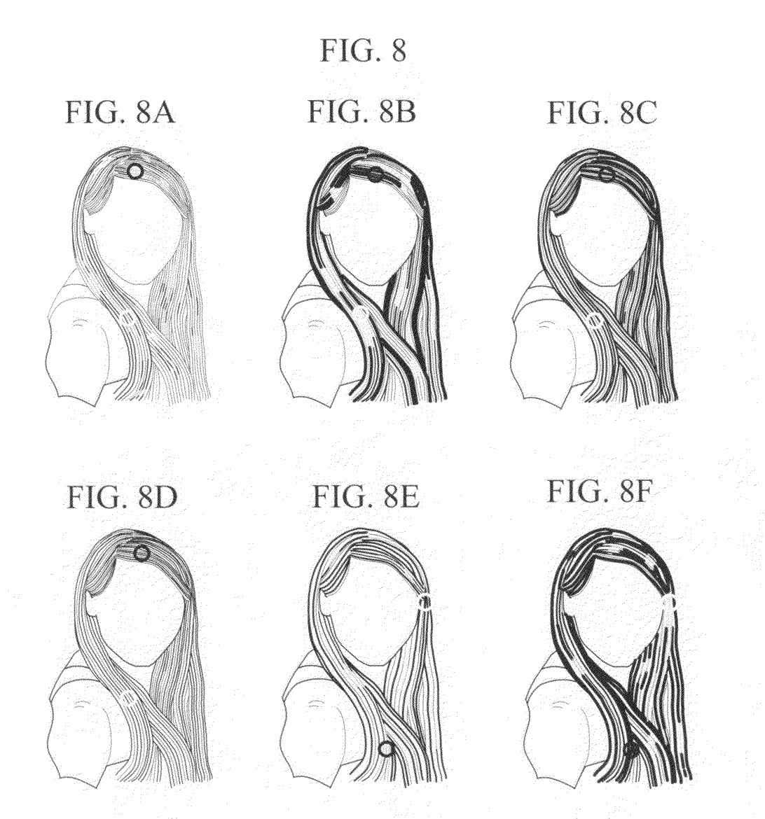

The discussion below of FIG. 5, FIG. 6, FIG. 7, and FIG. 8 demonstrates one possible approach to the second type of analysis and treatment. The current invention is not limited to this particular technique, and the tools of image capture under various lighting conditions, image analysis, and controlled agent deposition are useful for a wide range of techniques for hair color including random-looking appearance, enhancement of other natural patterns of variation, and the creation of artificial patterns of variation. These tools are also useful for the controlled deposition of agents other than hair coloring agents, or for the deposition of combinations of hair color and other hair agents.

In order to amplify or attenuate the visual effects of a signal along an axis of interest, a hair treatment substrate may be selectively applied to a localized region of interest in hair in agreement with or in opposition to the detected signal. Applying a hair treatment in opposition to or in agreement with measured optical characteristics in hair can be used to increase the signal to noise ratio, wherein the signal is considered any visual component that it is desirable to amplify (i.e. a set of colors or spatial frequencies) and the other visual components which should not be amplified are considered the noise. As an example, combing hair can be thought of as a technique which improves the appeal of hair by decreasing the noise associated with random orientation of hairs relative to one another. In a more complex sense, the optical characteristics of hair can be analyzed in terms of different spatial frequencies and different color intensities to quantify more subtle traits and/or ranges which can be classified as signal or noise along a particular axis of an image. Once establishing the desirable and undesirable signal ranges, there are two ways in which they can be modified relative to each other to enhance the visual effect of the desired characteristics. In the first approach, the desirable signal ranges can be increased to make them more visually detectable relative to other levels of detail in hair. In the second approach, the undesirable signal ranges can be suppressed to make the desirable signal ranges more visually evident.

For example, in one application of the current invention, used for the selective streaking of hair along different axes based on measured variations at different spatial frequencies, it is desirable to suppress the middle spatial frequencies on the axis which parallels the length of the hair during the application process. In this case, the noise is considered to be the middle spatial frequencies, while the signal is the high and low spatial frequencies. The high and low end cut-offs for this example application are 1/25^{th} inch and 0.4 inch, respectively. Therefore, the noise can be determined by performing a band-pass filter, though there are numerous other ways to process an image which isolates this frequency range from the rest of an image. Interestingly, these cut-off values are very similar to those found to be of interest in the image processing algorithms used for Transparent Beauty to distinguish a middle spatial frequency band, which is similarly treated as noise and suppressed as a means of providing a visual benefit. Once establishing the components of an image which are considered as noise and which are considered as the signal to be preserved or amplified, the device control software can determine where and how much hair treatment to deposit in order to increase the signal to noise ratio. This can be accomplished by applying a material which attenuates the noise, such as is done with Transparent Beauty's muting or morphing of undesirable middle spatial frequency skin features, or by applying a material which amplifies the signal. In the context of traditional hair color notation of L*a*b, this control is typically accomplished by controlling luminance L, which is the lightness, which is composed primarily of the green component channel and therefore the green channel is a good approximation of L for normal hair colors. Interesting effects can also be created by adjusting the color channel components a or b as part of the correction strategy, for example to make the hair redder or more grayish. (This strategy of controlling the L channel by sensing only the green channel provides a practical benefit of reserving the red channel for other tasks such as normalization or fluorescent dye detection.)

In the same application of the invention, but along the axis which measures the optical characteristics across hairs during an application process, different sets of spatial frequencies are treated as the noise and the signal. Along this axis, it is beneficial to manage the signal within the middle spatial frequency range similar to how an equalizer is used in electronics. If there are natural patterns detected in the hair which are too weak relative to the other detected patterns, then it is beneficial to amplify them. However, if there are patterns which are too strong relative to other detected patterns, it is beneficial to suppress them. More specifically, the middle spatial frequency band can be split into two components, the high-middle frequencies (HMF) and the low-middle frequencies (LMF). In one approach of measuring the variation on the axis across hairs, the optical characteristics are analyzed and a decision regarding which characteristics to enhance and which to suppress is made by attempting to establish an approximate equality of high-middle spatial frequency variation and low-middle spatial frequency variation.

Note that the embodiment described here does not require the use of traditional hair dyes which alter the underlying color of hair. Rather the effects of enhancing or suppressing an existing signal can be achieved by simply applying an agent which lightens or darkens the hair without altering its hue. Other effects can be created by adjusting the hue or both the hue and intensity, but again these effects may be based on very modest additions to natural or current hair color.

### Damage to Hair:

### Disadvantages of Conventional Hair Coloring Techniques

Another disadvantage of conventional hair treatment techniques, particularly the use of permanent hair coloring, is that they often cause irrevocable damage to the hair. To date, permanent hair dyes require exposing the hair to an oxidative chemistry which damages the cuticle, the outer layer of the hair. The cuticle must be damaged as a way of allowing the dye precursors to penetrate into the hair shaft before they react and form a single, larger dye molecule. The damaging of the cuticle is also necessary in order to allow an oxidizing agent, most commonly hydrogen peroxide, to enter into the hair shaft and break down the color producing melanin molecules.

Damage to the cuticle resulting from the use of a permanent hair colorant has several effects on the hair. For example, damage to the cuticle causes hair to become more absorbent as a result of the breaks to the hydrophobic outer sheathing on hair. As the hair become more absorbent, its physical properties are altered. Also, oxidative hair dyes tend to make the hair more fragile and more inclined to produce split ends. Finally, the damage inflicted on the cuticle during an oxidative hair coloring process causes the hair to develop different reflective properties. Damaged hair generally has less shine because the damage cause light to reflect more diffusely, whereas undamaged hair has a more uniform surface and therefore reflects light more unidirectionally.

While conditioning agent can be used which bind to the cuticle of the hair, particularly at the sites where damage has occurred, and have a smoothing effect which serves to minimize the effects of these problems, it is merely a partial and temporary solution to a permanent problem. Because conditioners bind to the cuticle of the hair, they tend to wash out after just a few washings to the hair with shampoo. Also, the application of excessive conditioning agents can leave the hair weighted down, ultimately replacing one problem with another. (Another application of the current invention, however, is to address this issue by applying conditioner selectively, as required, through a more routine hair care regimen.)

Although there are semi-permanent and demi-permanent hair coloring systems available which do not necessarily require the use of an oxidative chemistry, customers must decide between the trade-off of permanence of color versus damage to hair and the additional expense associated with more frequent reapplication.

### Advantages of Selective Deposition

One embodiment of the current invention serves to greatly reduce the extent of damage to hair during a hair coloring treatment without necessarily sacrificing the permanence of the colorant applied. Because the invention allows for the optical detection of a localized region of hair and precise deposition of substrates within that region, it is possible to use the applicator to use an oxidative hair coloring system more acutely. Even without changing the chemical components of the hair coloring system, the amount of damage could be reduced using selective deposition in two ways. First, the total amount of material applied can be minimized, thus reducing unnecessary oxidation of the cuticle. Second, the optical detection potential of the invention may be used to locate and deposit colorant on only specific regions of the hair which it is desirable to color, such as drastically different colored hairs, hair roots, or more subtle middle spatial frequency regions along an axis of the hair.

In an alternate embodiment and application of the invention, the permanence of a hair colorant system used becomes less critical because the user is using colorant only to enhance pre-existing, natural variations in the hair, whether they are present as a result of either natural hair color or previous applications of a desired colorant. If a customer is selectively applying colorant to local regions of the hair based on the detection of underlying optical variations and patterns, the effects of fading which could result from the use of a semi-permanent dye become less visually evident. Therefore, in this application, hair coloring chemistries which are damaging to the hair may be avoided.

### Thoroughness of Application:

Problems with Existing Approach to Hair Coloring

For existing hair treatments, the thoroughness of application is a crucial factor in establishing a successful treatment. For example, it is important to color the entire head of hair in a single treatment session for several reasons. Particularly when coloring the hair using a traditional approach, and more particularly when the hair is being dyed a color which is highly differentiated from its color before a dye session, if the entire head is not treated in a single session, it is likely that there will be noticeable differences in the treated and untreated regions of hair. Also, prior art shows a lack of applicators available which could be used to accurately apply an adequately small quantity of a hair treatment such that a partial deposition would not be clearly visible. The current invention provides such a means of localized, selective deposition of small quantities of a hair treatment substrate. Finally, existing chemistries, particularly for at-home hair coloring, are typically packaged in bulk quantities which are intended to cover the entire head or large portions thereof. Such a form of packaging does not lend itself to only partial coverage.

In one embodiment and application of the current invention in which optical sensing is used to detect variations in hair at different wavelengths or spatial frequencies and make selective depositions of hair treatments accordingly as a means of enhancing or suppressing the measured characteristics, the difference in the hair before and after application is less obvious than is the case with traditional application techniques. For example, if it is desired to increase and/or decrease natural color variations in hair at set spatial frequencies using a lightening and/or a darkening agent, there is less change in the hair after the application process than typically occurs using conventional dye processes. As a result of the limited change required for visual benefit when using the current invention to locally detect and selectively apply a hair treatment, it is less critical that the entire head be treated at one time. Therefore, one substantial benefit of the current invention is that it allows for the strategic placement of minor variations such that there is a visual improvement without an obvious indication of where treatment has been applied. This is similar to an important aspect of Transparent Beauty, in which the controlled deposition on skin may be used as a random and partial deposition device without leaving obvious indications of “edges” where material had and had not been applied. In effect, the current invention reduces the constraint of whole head applications for conventional hair treatment techniques.

Another important challenge associated with the thoroughness of conventional hair treatment techniques revolves around the difficulty ensuring that the hair treatment material is thoroughly distributed to all desired regions of the hair. The optical sensor of the current invention may be used to measure the amount of material locally deposited in a given region of hair, on multiple axes. In one embodiment, a fluorescent tracer material may be included with the colorant which serves to optically provide feedback regarding where and how much hair treatment material has been deposited. This material would be intended not to bind to the hair during the treatment process.

Also, in the case of a hair coloring application of the current invention, the chemical components of a colorant system may be mixed as needed, as a part of the deposition process, by combining portions of multiple precursors in an appropriate ratio. This approach reduces the total quantity of product applied and allows for a more systematic deposition of colorant, aided by user feedback via the amount of detected fluorescent tracer locally detected by the applicator device.

### Durability:

### Unmet Needs of Convention Hair Treatments

Durability of conventional hair treatments, particularly for hair coloring, is another area with significant trade-off. The less damaging semi-permanent and demi-permanent hair coloring systems are less durable that permanent dyes. The fading color of these dyes is significant when considered along with the time and expense required to treat hair using conventional techniques. Noticeable color changes can occur between treatments, but customers are disinclined to color their hair more frequently due to the inconvenience and cost of conventional approaches. Conversely, the more permanent coloring systems offer less color variation over time, but are more damaging to the hair, as has been previously discussed.

### Benefit of Current Invention

As a result of the fact that only small quantities of hair colorant chemistry are deposited, the current invention offers a potential to color hair using a less permanent dye on a more frequent basis without long deposition processes or cost increases. As discussed above, in an embodiment that uses the invention to identify optical characteristics of interest in hair, this point is particularly strong because little change is needed to provide visual benefit. With this embodiment, the ability to selectively modify pre-existing, natural variations also helps to minimize the visual changes which occur as a dye begins to fade. Also, the real-time analysis of localized sections of hair and the potential to deposit only small quantities of material help prevent a long application time. In one embodiment may be possible to deposit a colorant formulation in an appropriate quantity such that the customer is not required to rinse their hair after application.

### Speed of Application:

### Approximate Times of Conventional Hair Treatments

The speed of a hair treatment application is another area which can benefit from the current invention. Using conventional treatment methods, in order to obtain more natural visual patterns in hair, customers often spend from one to four hours at a salon. Even with these long application times, the final appearance generally lacks the degree of natural variations along multiple axes and at multiple spatial frequencies present in untreated hair. Also, at-home hair treatments, specifically hair coloring systems, require initial mixing, application of the substrate(s), a waiting period of roughly 10 to 45 minutes to allow the chemical reactions and thus adequate treatment to occur, and finally a rinsing of excess chemicals.

### Potential Speed of Current Methodology

In one embodiment of the current invention, in which the optical measuring of local hair characteristics is used to determine where to selectively deposit small quantities of hair coloring chemistries, an application process may be performed much faster than is feasible using conventional coloring techniques. The fluorescent tracer component in one embodiment of the current invention enables the detection of where a hair treatment compound has already been applied, thereby allowing the customer to apply colorant in a more controlled manner with the help of user feedback based on the optical sensing of the applicator.

Also, as previously noted, the current invention facilitates a means of locally applying a hair treatment with a controlled deposition on a more frequent basis. One application of an embodiment for the current invention allows the device to be used on a daily or weekly basis as a replenishing or touch-up system. Under these circumstances, it becomes less critical to cover the entire head in a single application session, therefore reducing the necessary time for providing the hair treatment.

Finally, because the current invention involves local deposition of minute amounts of material at a time, it may be further capable to eliminate the need to rinse the hair after the application of a treatment product, such as a hair dye. If a semi-permanent or demi-permanent hair coloring system deposited using the current invention, the localized deposition can effectively reduce the amount of excess product deposited to the point that it becomes unnecessary to rinse the hair after treatment. This use of the invention can be aided by the ability to ensure that all of the deposited material binds to the hair cuticles.

### Roots

Problems with Conventional Hair Treatments

Following an application of a conventional hair coloring system, roots become increasingly visible with time, especially if the colored hair is highly differentiated from the natural hair color. As hair grows after the application of a hair dye, a definite line can be perceived between the treated hair and untreated hair roots. Although there are some applicators which are designed in an attempt to address this problem by allowing the coloring of only hair roots, they merely help direct the colorant towards the scalp. The current invention, however, can provide feedback regarding where the material is actually being deposited as well as a more localized, more controlled means of deposition at the hair roots.

### Benefit of Current Methodology

In the previously discussed embodiment in which the hair is analyzed to reveal patterns and variations within different spatial frequency bands as a means of determining where and how much material to deposit, the problem of distinct, differentiated color hair roots growing out is diminished. By enhancing and/or diminishing the visual effects of measured, natural variations in hair, untreated hair roots are less distinguishable from the rest of the hair than is the case when the entirety of hair is dyed and thus changed from its natural color. Also, if the current invention is used more frequently than conventional hair treatment techniques, then an additional benefit, aside from those already discussed, is that the problem of roots growing out is greatly reduced because they are much shorter and thus less noticeable. Finally, for the application of one embodiment previously described in which the methodology described uses the color over a section of hair on a given axis, the applicator would continually attempt to fade the color difference between the untreated roots and treated outer hair. This prevents a definite line from being produced, which thereby decreases the visual impact of hair roots growing in after an initial application of hair dye.

### Expense of Application:

Problem with Conventional Approach

The global nature of the application of hair treatment substrates, a large excess of material is deposited and then removed without affecting the hair. These excesses result in greater expense of chemical formulations, an added cost which could be greatly reduced with the current invention given the minimal amount of product required to treat hair. In order for a hair treatment to work effectively, it is typically necessary for the hairs to be coated with the desired compounds, however, the presence of material beyond this coating is largely ineffective and financially inefficient.

### Advantages of Selective Deposition

The use of selective deposition can significantly reduce the amount of excess material applied, thus reducing unnecessary costs of chemicals.

### Precise Application of Heat

In one embodiment of the current invention, heat can be locally applied a region of hair or a compound before it is locally deposited in the hair. Conventional hair treatment techniques would not support the application of heat beyond a nominal amount due to the potential for harm or discomfort to a customer. However, due to the minuteness of quantities of material deposited and the preciseness of application of the current invention, it is possible to apply heat as part of the deposition process without causing the level of irritation that would result from the more global application techniques.

The application of heat with the current invention offers the benefit of catalyzing reactions. For example, during a hair coloring application, the application of heat in combination with the coloring substrates could serve numerous purposes. The heat could speed the reaction, thus reducing the application times. Also, heat can be more easily controlled in hair than a wet chemistry, so the heat could be more precisely directed to catalyze a reaction at specifically desired locations which are difficult to isolate with a strictly chemical deposition. Another benefit that heat could allow is for the applied chemistry to be less concentrated and/or less damaging to hair. The application of heat to the chemistry would allow the necessary reactions to have the needed energy, but without the heat, the damage to hair could be greatly reduced.

### Control Parameters to Enhance or Mimic Desirable Variations in Hair Color

FIG. 5, FIG. 6, FIG. 7, and FIG. 8 are images of various subjects and images derived from Photoshop™ simulations of one hair treatment technique that is enabled by control strategies and devices of the present invention. These simulations are representative of a wide range of image analysis techniques which may be used to develop target hair agent deposition strategies with digital application devices.

One aspect of these representative simulations is that a dramatic improvement in hair appearance is possible without changing a subject's natural hair color. Rather than bleach and recolor a person's hair, this technique provides a combination of smoothing variations along the length of hair strands, and then amplifying desirable variations across strands.

These simulations represent different control strategies for two axes of hair strands—along and across. Deposition devices permit a further differentiation in a third axis-through strands at different depths of the hair.

FIGS. 5A and 6A are images of test subjects before any simulated treatment.

FIGS. 5C and 6B are images of test subjects after a simulated “COMB and STREAK” treatment as described below.

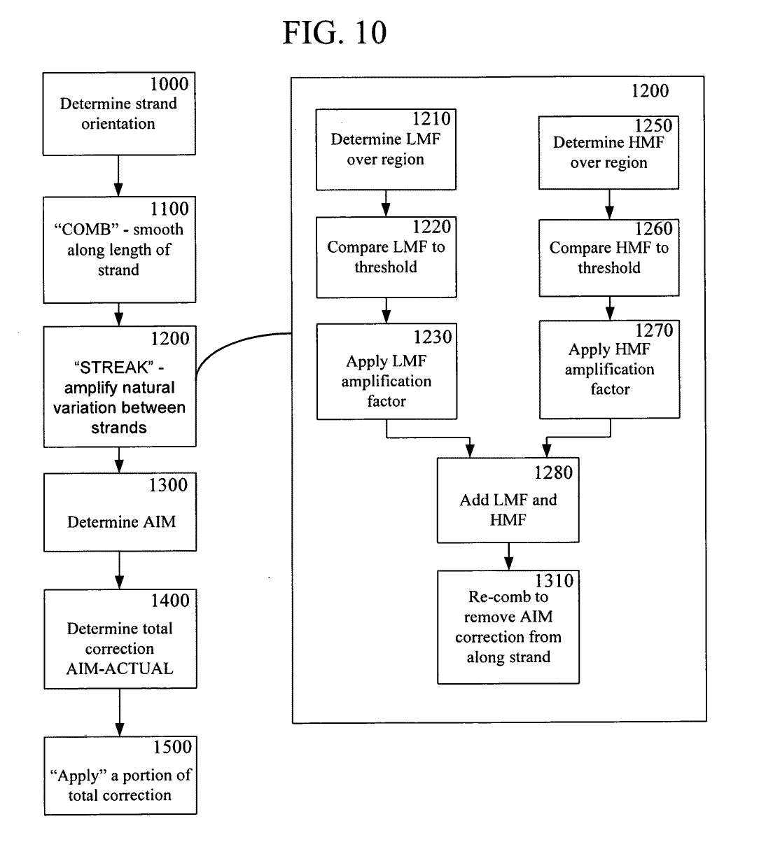

FIG. 10 is a flow chart that illustrates the simulation methods for FIG. 5, FIG. 6, FIG. 7, and FIG. 8.

At step 1000, the orientation of the strands in an image is determined. Note that in an application device, this step is straightforward when a brush or comb aligns hair so that the length of the strands is parallel to the direction of movement of the device, and strands are separated by tines. In the Photoshop simulation, strand orientation was determined by performing a directional blur in 5 degree increments. The orientation with the least blur was selected as the strand orientation.

At step 1100, a “COMB” operation was performed to smooth the strands in the direction of the strand orientation. This operation performed a smoothing over regions of 0.4 inch length along the hair strands.

FIG. 5B shows the simulated effects of only a “COMB” smoothing operation along the length of hair strands. FIG. 5D shows a mask 906, amplified by 4× actual image, of a calculated correction to achieve the COMB smoothing, applied to FIG. 5A to achieve FIG. 5B.

At step 1200, a “STREAK” operation was performed to identify and amplify desirable natural middle frequency variations between strands, that is perpendicular to the strands.

This streak operation was initially performed by a single bandpass filter operation for the entire middle frequency spectrum of 1/25 (0.04) inch to 0.4 inches. Those results were disappointing in that the treatment of some subjects had an artificial appearance. A more detailed analysis of those results indicated major differences between subjects and in some subject's own hair between the lower middle frequency (LMF) band of ⅛ (0.125) inch to 0.4 inch, and the high middle frequency (HMF) band of 0.04 inch to 0.125 inch. A much more pleasing result was obtained by analyzing and processing the LMF and HMF bands separately and then adding the results.

The natural frequency components of the subjects' hair are shown in the high middle frequencies in FIG. 8A and in the low middle frequencies in FIG. 8E. In both figures, the black circles 1802 show areas with low natural energy appearing as a flat grey. Also in both figures, white circles 1804 show areas with high natural energy appearing as black and white streaks. These white and black circles are extended across all FIG. 8 illustrations at like positions.

FIG. 8B illustrates directly the energy level at each point in the high middle frequencies. Note that the area under the white circle is white and the area under the black circle is dark. FIG. 8F is the equivalent to FIG. 8B for the low middle frequencies. Note that the areas that are strong in one frequency band are not necessarily strong in the other frequency band.

FIG. 8G attempts to equalize the energy of low middle frequencies in all positions. Note that the area in the white circle of 8E, which already had sufficient energy as technically shown by the white area under the white circle of FIG. 8F, has been left substantially unchanged in FIG. 8G. On the other hand, the area under the black circle in FIG. 8E has been shown by FIG. 8F to be deficient in low frequency energy content by the black region, and therefore the detail under the black circle in FIG. 8E has been contrasted up to yield the result in FIG. 8G. Therefore, FIG. 8G should have close to equal energy at all points in the low middle frequencies.

FIG. 8G now represents a goal to achieve beauty. Part of this goal has already been reached naturally, as shown in FIG. 8E. Therefore, to achieve the visual appearance of FIG. 8G it is necessary to deposit lightening or darkening agents only in proportion to the difference between the desired result in FIG. 8G and the natural starting point in FIG. 8E. The resulting deposition pattern, the difference between FIG. 8G and FIG. 8E is shown in FIG. 8H. Note that because this supplements, rather than countermands, natural streaking, less color change is needed to achieve a desired result.

FIG. 8C and FIG. 8D are the equivalents of FIGS. 8G and 8H for the high middle frequencies. The actual deposition of lightening or darkening coloring agent is deposited in proportion to the sum of FIG. 8D and FIG. 8H. In particular, a lightening agent may be applied when the sum of FIG. 8D and FIG. 8H is lighter than neutral grey and a darkening agent may be applied when the sum is darker than neutral grey. As an example, where FIG. 8D and FIG. 8H are both light, a lightening agent may be deposited. Where both are dark, a darkening agent may be applied. And when one is light and one is dark, very little may be applied, depending on which is stronger. However, the lightening and darkening agents do not need to be applied at the same point.

By way of further illustration, FIG. 7A is a magnified version of FIG. 8C, illustrating the aim for the high middle frequencies. FIG. 7B is a magnified version of FIG. 8G, illustrating the aim for the low middle frequencies. FIG. 7C is the sum of the hair streaking in FIG. 7A and FIG. 7B, and represents the overall goal to achieve beauty.

FIG. 6A shows an original image of the subject. FIG. 6B shows the simulated appearance of the subject after practicing the invention, both for the comb effect and for the streaking effect. Note that the smooth, well-defined strands make hair appear healthy and attractive.

One aspect of the current simulation method is to identify deposition device control strategies which identify desirable high levels of energy, preserve those naturally attractive areas, and identify specific opportunities to increase either the LMF, the HMF, or both, in order to achieve high levels of energy throughout the hair.

At step 1210, the LMF energy level for a region is determined. At step 1220, the LMF energy level is compared to a desired threshold. In this example, that threshold was selected by beauty consultants based on subjective analysis of hair beauty. In production devices, the threshold is likely to be pre-set, user selectable, or calculated based on observed characteristics of the user's hair. By analogy, the Eraser Brush skin device may either be preset or may calculate thresholds based on the user's average skin characteristics. At step 1230, an amplification factor was applied to the region if the LMF energy was below the threshold. In this example, the preferred amplification factors were in the range of 2× to 3× of the difference between the actual and threshold values. However, at step 1500 this amplification was subsequently de-rated to 67% of the calculated correction.

At step 1250, the HMF energy levels for a region are determined. The region size for calculating the HMF energy levels is smaller than the region size for calculating the LMF energy levels. At step 1260, the HMF energy level is compared to a desired threshold. In this example, that threshold was also selected by beauty consultants based on subjective analysis of hair beauty. At step 1270, an amplification factor of 2× to 3× was applied to the region if the HMF energy was below the threshold.

At step 1280, the LMF and HMF corrections are added.

The selection of the two LMF and HMF bands is one method of finding more discrete enhancements. In other examples, three or more bands within the middle spatial frequencies may be considered individually. The treatment protocols are not limited to middle spatial frequencies, and some approaches which have positional awareness can impose low frequency variations over the hair, or superimpose these low frequency variations with middle frequency strategies. High frequency treatment schemes are also possible with either precise control such as inkjet delivery system, or delivery systems which incorporate the ability to divide hair into small strands or individual hairs.

In the simulation, these corrections were performed in a radius because of limitations in Photoshop. Therefore, in addition to amplifying features on the strand of interest, an undesired adjustment to neighboring strands was also performed. Rather than conduct more extensive simulation programming, the radial effects were removed by performing a second COMB operation at step 1310. In a device, tines again provide the directional orientation and strand separation that permit independent strand treatments without this second COMB operation. In the device, the COMB smoothing and STREAK amplification operations may be performed simultaneously as the device is moved over or through the hair.

In the two-band analysis approach of FIG. 10, the AIM or target correction of step 1300 was determined by adding the band corrections at step 1280.

The desired total correction at step 1400 is then the difference between the actual image energy level and the AIM levels.

At step 1500, the simulation “applies” a portion the desired correction. In this example, the portion was selected to be 67%. In practice, there are two reasons to under-correct. First, each treatment session is typically multiple pass over portions of the hair, so that it is desirable to allocate only a portion of the overall correction on a single pass. Second, in practice, the device is likely to be used frequently, so that modest corrections are desired for any particular session.

This example simulation was conducted in two dimensions. Devices will have the capability for three dimensional treatment strategies as hair orientation elements such as combs permit separate treatments for the along-strand, across strands, and depth axes.

These simulations adjusted only the lightness, or conventionally stated the “L” component of the L*a*b color space, of the existing hair color. Significant improvement in appearance was obtained by this magnitude-only adjustment. Since many of the deposition devices, such as multi-component spray or inkjet devices permit the controlled deposition of multiple agents, applicants expect different, and perhaps more dramatic results from the selective simulation and application of various coloring agents, or various combinations of coloring agents, darkening agents, lightening agents, gloss agents, or body agents.

### Digital Brush

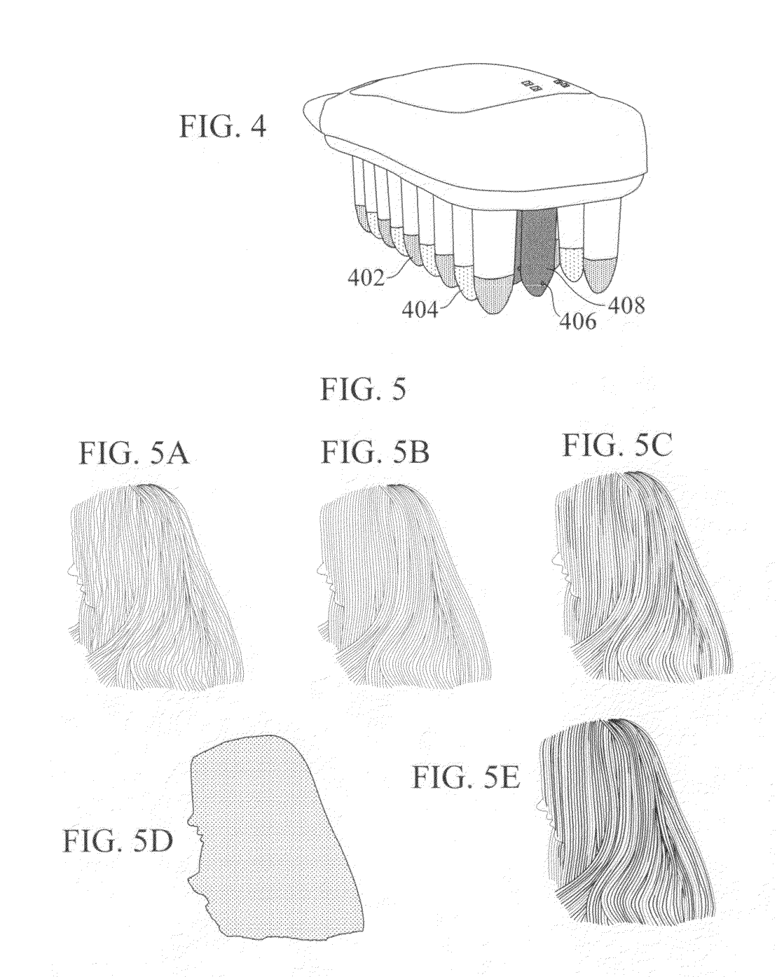

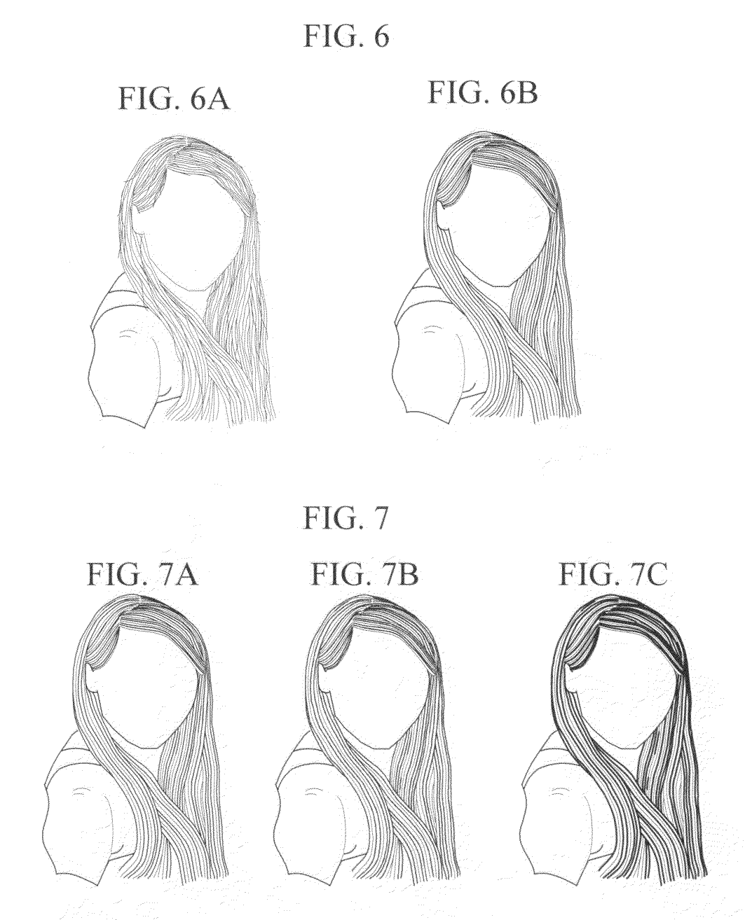

One embodiment of the current invention combines image analysis and deposition control strategies, such as described above, with a “Digital Brush” application device as represented by FIGS. 3 and 4 and more fully described in copending U.S. Provisional Patent Application No. 61,271,512. That device is one example of a “tine delivery device” where one or more hair agent is selectively delivered to hair through a comb or brush device. Other types of tine delivery devices are described below, and other devices for the selective deposition of hair agents may be based on inkjet, spray, or other devices as described in the referenced Transparent Beauty and Eraser Brush patent applications.

For example, FIG. 3 illustrates an embodiment of a digital brush showing primary components such as a power supply 302, processor 604, a substrate reservoir 616, and a compressed gas cylinder 304.

In another example, FIG. 4 illustrates an embodiment of a digital brush with separate emitting tines 402, sensing tines 404, deposition tines 406, and an independent deposition pathway 408.

### Agent Detection of Multiple Pass Operation

Many embodiments of the current invention are on multiple passes over or through hair, so that only a portion of a desired overall correction is applied in a single pass.

Copending U.S. Provisional Patent Application No. 61,271,512 also describes an apparatus and method for automatically local detection of either the presence or absence of hair colorant or the relative amount of hair colorant present. The detection of hair colorant is accomplished by means of the evaluation of characteristics having an established relationship with the amount of hair colorant present. Examples of such characteristics include fluorescence, pH, and inductance. With each possible characteristic for locally analyzing the presence and/or amount of colorant in a portion of hair, the information obtained from a sensor is interpreted by means of an integrated circuit which, in turn, provides signals and/or user feedback as a means of controlling further deposition of colorant. This process allows for a dynamic deposition control which helps to minimize excessive chemical treatment of hair while simultaneously preventing the user from accidentally having portions of the hair go untreated.

One aspect of the current invention is the ability to base hair image analysis on a first channel, such as the green channel, and to use a second channel for the detection of indicators.

### Medical Applications for Optical Emitting and Sensing

Copending U.S. Provisional Patent Application No. 61,271,512 describes the use of a digital brush device for use as a platform for medical and/or health related observations derived from hair analysis. These observations, and changes over time, may provide indications of breast cancer, lung cancer, prostate cancer, colon cancer, insulin dependent diabetes mellitus, and Alzheimer's disease. In various embodiments, a hair care device can provide this medical monitoring capability, such as with fluorescent dyes and infrared analysis, in conjunction with the device's normal use as a hair treatment device. This capability is analogous to the use of Transparent Beauty and Eraser Brush devices for monitoring skin changes over time.

### Digital Comb

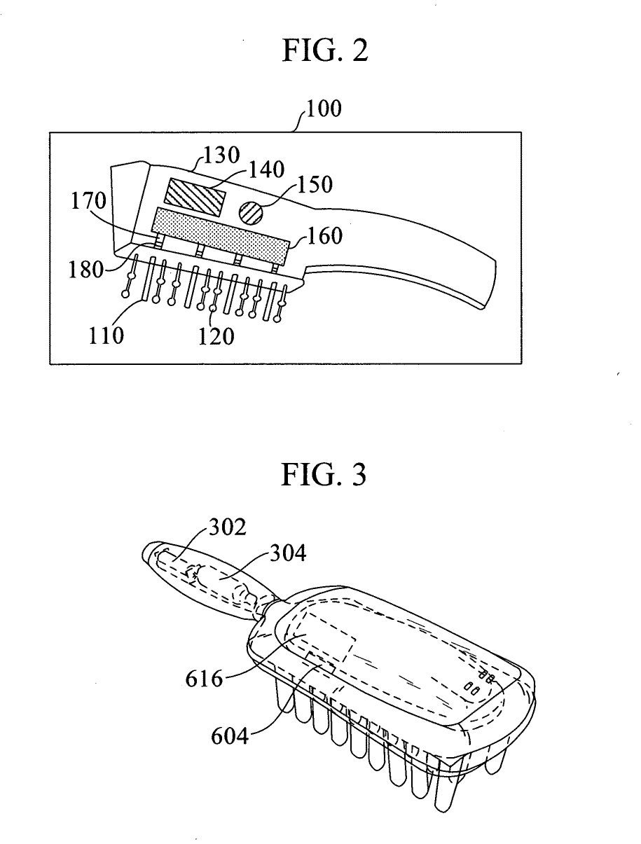

U.S. Provisional Patent Application No. 61,012,473 describes an apparatus and method for use as a digital comb that automatically senses the lightness or darkness of a person's hair color and the lightness and darkness of hair roots. The digital comb then deposits one or more hair colorants, such as dye, pigment, or bleach, precisely onto the specific hair roots that need to be darkened or lightened. Typically the digital comb is used with a colorant that is specific to the user's overall hair color, for example to blond, brown, black, red, grey, or white hair. Users simply move the digital comb through their hair multiple times, in the common combing motion, and the digital comb automatically lightens or darkens their hair roots until enough of the hair colorant has been deposited to color the hair roots correctly. FIG. 2 illustrates one embodiment of the digital comb, the teeth of which comprise tubes 110 and guides 120. In this example, four tubes 110 are used, with interstitial guides 120 with spherical tips to avoid scratching the skin. The body 130 of the comb 100 contains optical and mechanical means to sense the need for coloring enhancements and to automatically deposit hair colorant.

The tube design allows the transfer of a hair colorant to be deposited at the base of the tube, under computer control. A fiber optic cable transfers light to the base of the tube, and returns as a continuous cable. Most of the fiber optic cable is sheathed, shown in green. However, at the base, a section of the fiber optic cable is unsheathed, and the unsheathed section may be wrapped around the tube there. This configuration allows the unsheathed section to touch hair roots and break the total internal reflection of the fiber optic cable.

### Digital Comb with Multiple Levels of Sensors

U.S. Provisional Patent Application No. 61,056,282 describes another tine delivery device where the tines comprise tubes and guides. In one example, five tubes are used with intermediate interstitial guides. The tines may include spherical tips to avoid scratching the skin. In other embodiments, the comb may be comprised entirely of tubes or have fewer tubes and more interstitial guides. Electrical emitters and sensors for measuring conductance or inductance are mounted at one or more specific levels above the tip on the inside faces of the outer surface of the tines so that an emitter and sensor are opposite each other on adjacent tines (an emitter-sensor pair). An embodiment of a tube allows the transfer of a hair colorant to be deposited at the base of the tube, under computer control. The body of the comb contains electronic and mechanical means to sense the need for coloring enhancements and to automatically deposit hair colorant. When the device is turned on, the processor continuously measures the conductance or inductance at each emitter-sensor pair. When there is no hair between an emitter and a sensor pair on adjacent tines (an emitter-sensor pair), there is no conductance and therefore a zero reading. When there is dry hair between the emitter and sensor pair on adjacent tines, there is a mid range reading and a signal to deposit is made. When hair coated by a colorant is between the sensor and emitter, there is the highest conductance, which could indicate that no deposition should be made. The amount of conductance will vary depending on the amount of colorant applied.

### Digital Brush with Telecentric Image Capture

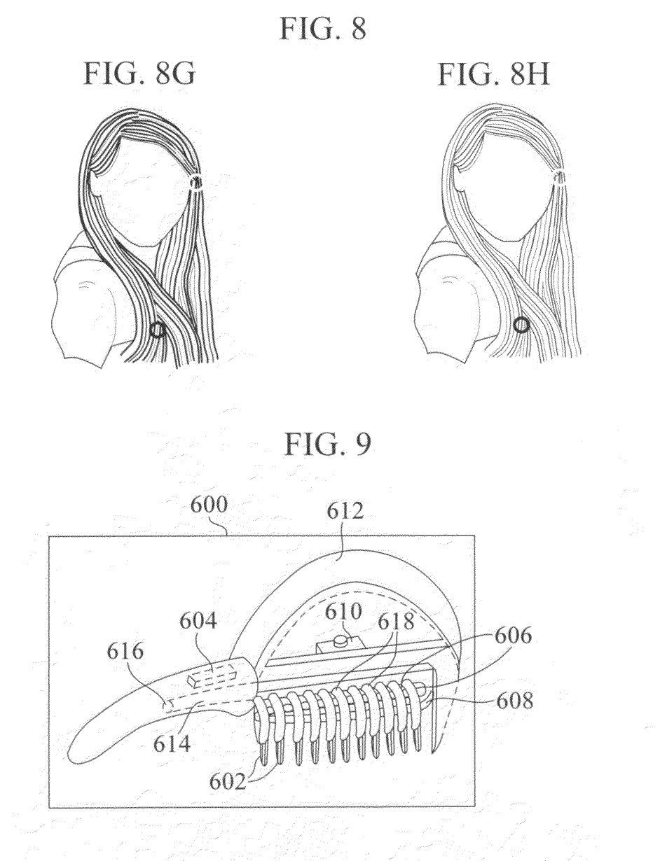

Another embodiment of the Digital Brush is shown in FIG. 9. In this embodiment, the Digital Brush 600 comprises tines 602, as on any comb. When the Digital Brush 600 is moved through the hair, these tines 602 can separate strands of hair along the x or y axis of the head. Fine tines 602 with ordinary spacing may be used, since they move through the hair easily and tend not to move colorant around the hair.

As the Digital Brush 600 is moved through the hair, a processor 604 controls the Digital Brush's 600 operations. LEDs 606 illuminate the hair just behind the tines 602, their light passing through a polarizing filter 608 that reduces reflections to improve accuracy of measurements of brightness and color. In an embodiment, a large number of LEDs 606 may be used, for example 30-40, to provide light that is as diffuse and uniform as possible, as in a line radiator. In another embodiment, fewer LEDs 606 may be used, for example five, and directed with mirrors to provide diffuse and uniform light. In still another embodiment, at least two rows of LEDs 606 may be used, again to diffuse and uniform light.

In an embodiment, green LEDs 606 may be used as advantageous sources for capturing data about the hair. In another embodiment, ultraviolet LEDs 606 may be interspersed with the green LEDs 606, to illuminate tracer material contained in the colorant.

A camera 610 captures images of the hair that are reflected in the underside of a curved mirror 612 mounted above the camera 610 and focused on the hair just behind where the tines 602 have moved, the mirror 612 providing a tele-centric view of the hair. The light reaching the camera may also pass through the polarizing filter 608 to reduce reflections.

Pressurized hair care compound 614, for example a colorant, is stored in a reservoir 616 in the handle of the brush 600. According to enhancement calculations made by the processor, the hair care compound 614 in deposited onto the hair through a row of deposition tubes 618, which may be mounted approximately ¼ of an inch back from the tines.

The deposition tubes 618 may deposit at the surface of the head of hair, or they may be lengths suitable for depositing at the surface and at lower levels in the hair.

### Modified Eraser Brush Device

In one embodiment of the current invention, a common base unit is used for the application of compounds to skin and to hair. The base comprises different attachments and control settings for each of the different applications, but the major hardware components are shared between for each use of the selective analysis and deposition device. In one example, a handheld Eraser Brush device has a removable comb attachment for orienting hair strands; a control mode is selectable between skin and hair treatment; and different cartridges are provided for hair and skin. It is possible that common chemistries can be developed so that a single set of cartridges can be used for both skin and hair.

### Tine Delivery Device with Photodetector

While most embodiments described above describe camera applications, it is also possible to develop hair devices which work with simple LEDs and photodetectors as described in the Eraser Brush patent application.

1. A device for improving hair appearance comprising:
(a) at least one power source;
(b) a reservoir for a hair care compound;
(c) a processor operatively connected to the at least one power source and the reservoir for the hair care compound;
(d) a light emitter operatively connected to the at least one power source;
(e) at least one optical sensor operatively connected to the processor; and
(f) a deposition pathway operatively connected to the reservoir for the hair care compound and located adjacent to at least one tine.

2. The device of claim 1 wherein the light emitter is a plurality of light emitting diodes (LEDs).

3. The device of claim 2 wherein the light emitted from the plurality of LEDs passes through a polarizing filter.

4. The device of claim 1 wherein the at least one optical sensor is a camera and a mirror is mounted above the camera.

5. The device of claim 1 wherein a hair care compound is located in the reservoir for the hair care compound and the hair care compound contains a fluorescent tracer material.

6. The device of claim 5 wherein the fluorescent tracer material has an excitation peak falling within one of an ultraviolet wavelength and a blue wavelength and an emission peak falling within one of a green wavelength, a red wavelength and an infrared wavelength.

7. A method for improving hair appearance comprising the steps of:
(a) illuminating a region of at least one strand of hair with a light emitter;
(b) sensing a spatial frequency in the region of the at least one strand of hair with an optical sensor;
(c) analyzing the spatial frequency in the region of the at least strand of hair; and
(d) depositing a hair care compound based on results from analyzing the spatial frequency in the region of the at least one strand of hair.

8. The method of claim 7 wherein depositing the hair care compound includes depositing a fluorescent tracer material.

9. The method of claim 7 wherein the spatial frequency is an energy level measured in the region of the at least one strand of hair over distances in the range of 0.04 to 0.40 inch.

10. The method of claim 8 further comprising:
detecting emission wavelengths of the fluorescent tracer material to ascertain whether an appropriate amount of the hair care compound has been deposited in the region of the at least one strand of hair.

11. The method of claim 7 further comprising:
suppressing the spatial frequency in the region on a first strand of hair and enhancing the spatial frequency in the region of a second strand of hair adjacent to the first strand of hair.

12. The method of claim 7 wherein depositing the hair care compound based on results from analyzing the spatial frequency in the region of the at least one strand of hair is done in agreement with the spatial frequency in the region of the at least one strand of hair.

13. The method of claim 7 wherein depositing the hair care compound based on results from analyzing the spatial frequency in the region of the at least one strand of hair is done in opposition to the spatial frequency in the region of the at least one strand of hair.

14. The method of claim 7 further comprising:
generating an image showing the spatial frequency in the region of the at least one strand of hair.

15. The method of claim 7 further comprising:
applying heat to the region of the at least one strand of hair before depositing the hair care compound.

16. The method of claim 7 further comprising:
applying heat to the hair care compound before depositing the hair care compound.

17. A method for improving hair appearance comprising the steps of:
(a) determining the orientation of at least one strand of hair;
(b) smoothing the at least one strand of hair in the direction of the orientation of the at least one strand of hair;
(c) identifying natural middle spatial frequency variations in the at least one strand of hair; and
(d) amplifying natural middle spatial frequency variations in the at least one strand of hair by depositing a hair care compound in an amount related to a total correction calculated from analysis of the natural middle spatial frequency variations in the at least one strand of hair.

18. The method of claim 17 wherein amplifying natural middle spatial frequency variations in the at least one strand involves determining a lower middle spatial frequency energy level over a region of the at least one strand; comparing the lower middle spatial frequency energy level to a threshold value; applying a lower middle spatial frequency amplification factor if the lower middle spatial frequency energy level is below the threshold value; determining a high middle spatial frequency energy level over the region of the at least one strand; comparing the high middle spatial frequency energy level to a second threshold value; applying a high middle spatial frequency amplification factor if the high middle spatial frequency energy level is below the second threshold value; and adding the lower middle spatial frequency amplification factor and the high middle spatial frequency application factor to ascertain a targeted correction for the region.

19. The method of claim 18 further comprising:
determining the total correction by calculating the difference between an actual image energy level for the region and the targeted correction for the region.

20. The method of claim 19 further comprising:
applying a portion of the total correction to the region.
